(19) United States
(12) Patent Application Publication
(10) Pub. No.: US 2026/0148525 A1
(43) Pub. Date: May 28, 2026

(54) DIFFUSION MODEL CONDITIONING ON MULTI-DOMAIN MEDICAL IMAGES WITH MISSING DOMAINS

(71) Applicant: Siemens Healthineers AG, Forchheim (DE)

(72) Inventors: Gengyan Zhao, Robbinsville, NJ (US); Youngjin Yoo, Princeton, NJ (US); Boris Mailhe, Plainsboro, NJ (US); Eli Gibson, Lawrenceville, NJ (US); Dorin Comaniciu, Princeton, NJ (US)

(21) Appl. No.: 18/961,660

(22) Filed: Nov. 27, 2024

(51) Int. Cl. G06V 10/77; G06V 10/82; G16H 30/40

(52) U.S. Cl. CPC G06V 10/7715; G06V 10/82; G16H 30/40; G06V 2201/03

(57) ABSTRACT

Systems and methods for performing a medical imaging analysis task conditioned on multi-domain medical images with missing modalities are provided. 1) one or more medical images each in a different domain and 2) a domain code defining a presence of the different domains in a set of predefined domains are received. One or more weights are determined based on the domain code. One or more parameters of a machine learning based encoder are updated based on the one or more weights. Features are extracted from the one or more medical images using the machine learning based encoder with the one or more updated parameters. A medical imaging analysis task is performed based on the extracted features. Results of the medical imaging analysis task are output.

### TECHNICAL FIELD

The present invention relates generally to AI/ML (artificial intelligence/machine learning) based medical imaging analysis, and in particular to diffusion model conditioning on multi-domain medical images with missing domains.

### BACKGROUND

Diffusion models are a type of generative AI model that generate data by simulating a diffusion process, which involves adding noise to data and then reversing the process to generate new data. Diffusion models have attracted attention recently due to their broad applicability. However, the applicability of diffusion models to medical imaging has been challenging due to the inherent complexity and heterogeneity of medical image data.

Different types of medical images provide different types of information. For example, CT (computed tomography) images more effectively capture bone, air, and blood contrasts, while T1, T2, and proton density-weighted MR (magnetic resonance) images more effectively capture tissue characteristics. However, conventional diffusion models typically accept only a single medical image as input for medical imaging analysis and are unable to utilize information provided by a plurality of medical images in different domains. In addition, the differences in acquisition protocols across clinical sites often result in the unavailability of medical images in certain domains. Conventional diffusion models are unable to account for the unavailability of such medical images.

### BRIEF SUMMARY OF THE INVENTION

In accordance with one or more embodiments, systems and methods for performing a medical imaging analysis task conditioned on multi-domain medical images with missing modalities are provided. 1) one or more medical images each in a different domain and 2) a domain code defining a presence of the different domains in a set of predefined domains are received. One or more weights are determined based on the domain code. One or more parameters of a machine learning based encoder are updated based on the one or more weights. Features are extracted from the one or more medical images using the machine learning based encoder with the one or more updated parameters. A medical imaging analysis task is performed based on the extracted features. Results of the medical imaging analysis task are output.

In one embodiment, the domain code further defines an absence of one or more domains of the set of predefined domains from the different domains. Each position of the domain code is associated with a respective one of the set of predefined domains. The one or more weights are determined by projecting the domain code to the one or more weights using a linear projector.

In one embodiment, a weight parameter and a bias parameter of the machine learning based encoder are updated. The one or more parameters are updated by determining a dot product of the one or more parameters of the machine learning based encoder and a respective one of the one or more weights.

In one embodiment, one or more all-zero tensors for one or more domains of the set of predefined domains absent from the different domains are received. The one or more medical images are concatenated with the one or more all-zero tensors. Features are extracted from the concatenation.

In one embodiment, one or more masks of at least one of a pathology or an organ are received. The one or more medical images are concatenated with the one or more masks. Features are extracted from the concatenation.

In one embodiment, the medical imaging analysis task comprises medical image synthesis.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a medical imaging analysis task, in accordance with one or more embodiments;

FIG. 2 shows a workflow for extracting features from one or more medical images for performing a medical imaging analysis task, in accordance with one or more embodiments;

FIG. 3 shows a network architecture of a DFN ControlNet conditioned on multi-domain medical images with missing domains, in accordance with one or more embodiments;

FIG. 4 shows a network architecture of a DFN DDPM/LDM conditioned on multi-domain medical images with missing domains, in accordance with one or more embodiments;

FIG. 5 shows an exemplary artificial neural network that may be used to implement one or more embodiments;

FIG. 6 shows a convolutional neural network that may be used to implement one or more embodiments;

FIG. 7 shows a data flow diagram for using a generative adversarial network that may be used to implement one or more embodiments;

FIG. 8 shows a schematic structure of a recurrent machine learning model that may be used to implement one or more embodiments; and

FIG. 9 shows a high-level block diagram of a computer that may be used to implement one or more embodiments.

### DETAILED DESCRIPTION

The present invention generally relates to methods and systems for diffusion model conditioning on multi-domain medical images with missing domains. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a novel image synthesis framework for conditioning diffusion models on multi-domain medical images with missing domains, enabling efficient handling of various multi-to-one medical image synthesis tasks. The image synthesis framework utilizes a DFN (dynamic filter network) to take advantage of the information offered by medical images of multiple domains and to dynamically adapt to missing domain scenarios, eliminating the need for training multiple models to manage different scenarios where certain domains are missing from the input medical images. Advantageously, the image synthesis framework maximizes the utilization of all available data, even in the presence of missing domains, while minimizing computational cost and training effort.

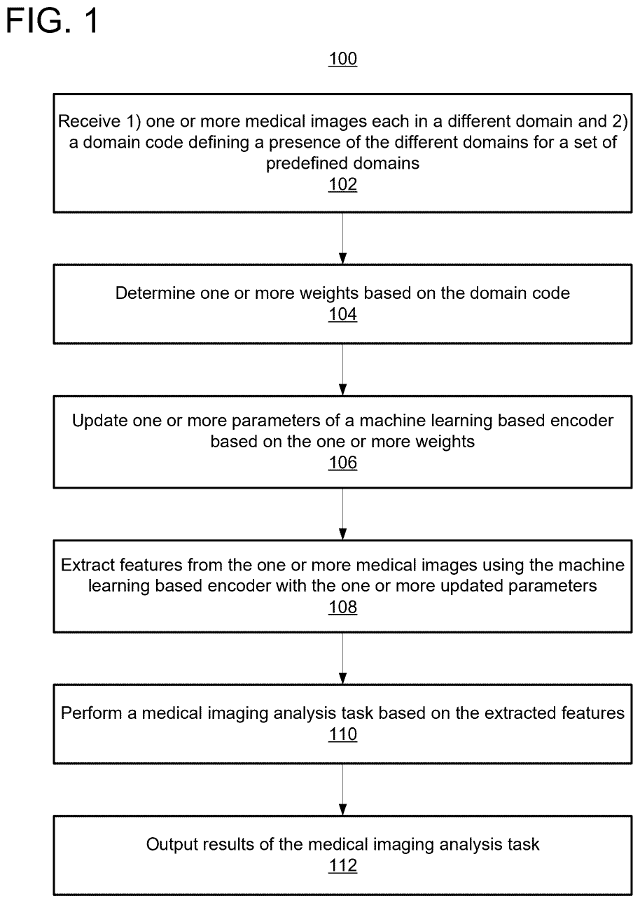

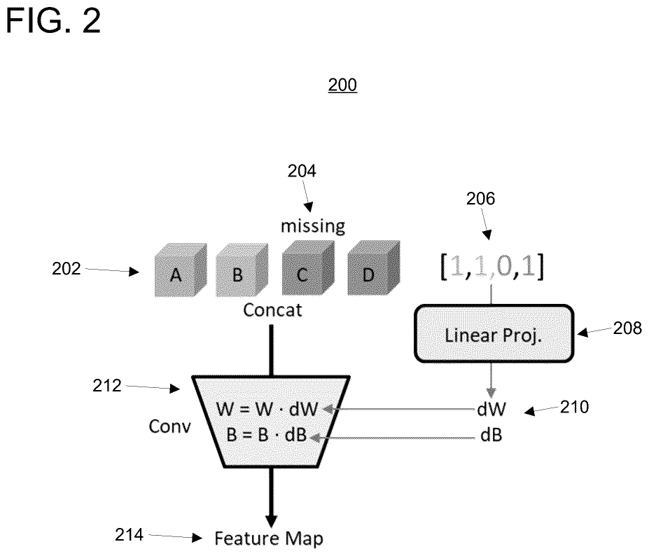

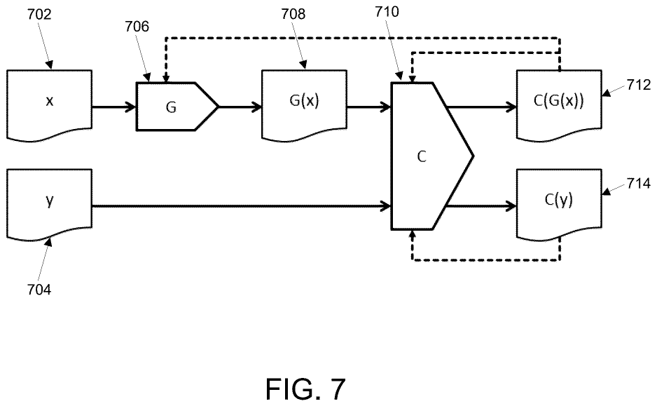

FIG. 1 shows a method 100 for performing a medical imaging analysis task, in accordance with one or more embodiments. The steps and sub-steps of method 100 may be performed by one or more suitable computing devices, such as, e.g., computer 702 of FIG. 7. FIG. 2 shows a workflow 200 for extracting features from one or more medical images for performing a medical imaging analysis task, in accordance with one or more embodiments. FIG. 1 and FIG. 2 will be described together.

At step 102 of FIG. 1, 1) one or more medical images each in a different domain and 2) a domain code defining a presence of the different domains for a set of predefined domains are received. The set of predefined domains represents the domains that a machine learning based encoder (utilized at step 108) is trained to process. In one embodiment, the domain code further defines an absence of one or more domains of the set of predefined domains from the different domains. An all-zero tensor may be received at step 102 for each of the one or more absent domains.

The domain code may be represented in any suitable form for defining the presence of the different domains in the set of predefined domains and/or the absence of one or more domains of the set of predefined domains from the different domains. In one embodiment, the domain code is a 1×n vector, where n represents the number of domains in the set of predefined domains. Each position in the vector is associated with a respective domain of the set of predefined domains. The value at each position may be defined, for example, by a one-hot, where a value of 1 defines the presence of an input medical image in the associated domain and a value of 0 defines the absence of an input medical image in the associated domain. Other approaches for encoding the presence and/or absence of medical images in the set of predefined domains are also contemplated.

In one example, as shown in workflow 200 of FIG. 2, the set of predefined domains is domains A, B, C, and D. The one or more medical images is medical images 202 in domains A, B, and D and the domain code is domain code 206. Each position of domain code 206 is associated with a respective one of the set of predefined domains. Accordingly, a first position of domain code 206 is associated with domain A, a second position of domain code 206 is associated with domain B, a third position of domain code 206 is associated with domain C, and a fourth position of domain code 206 is associated with domain D. As shown in workflow 200, a medical image in domain C is absent or missing from the input. An all-zero tensor 204 is received in place of the medical image in domain C. As such, domain code 206 defines a value of 1 for the first, second, and fourth positions to define a presence of medical images in domains A, B, and D, and defines a value of 0 for the third position to define an absence of a medical image in domain C.

As used herein, a domain of a medical image refers to the modality of the medical image as well as the protocol used for obtaining the medical image in that modality. The modality of the one or more medical images may include, for example, MRI (magnetic resonance imaging), CT (computed tomography), US (ultrasound), x-ray, single-photon emission computed tomography (SPECT), positron emission tomography (PET), or any other medical imaging modality or combinations of medical imaging modalities. The protocol used for obtaining the medical image may include, for example, acquisition sequences or techniques for acquiring a medical image, such as, e.g., T1-weighted, T2-weighted, proton density-weighted MRI images, contrast and non-contrast images, CT images captured with low kV (kilovoltage) and high kV, or low and high resolution medical images. Accordingly, the different domains may be completely different medical imaging modalities or different image protocols within the same overall imaging modality. The one or more medical images may be represented in the image space (e.g., as pixel or voxel values in spatial coordinates) or the latent space (e.g., as a lower-dimensional, compressed representation of the one or more medical images represented as a feature vector). The one or more medical images in the image space may be 2D (two dimensional) images and/or 3D (three dimensional) volumes.

The one or more medical images and/or the domain code may be received, for example, by directly receiving the one or more medical images from an image acquisition device (e.g., image acquisition device 714 of FIG. 7) as the one or more medical images are acquired, by loading the one or more medical images and/or the domain code from a storage or memory of a computer system (e.g., storage 712 or memory 710 of computer 702 of FIG. 7), or by receiving the one or more medical images and/or the domain code from a remote computer system (e.g., computer 702 of FIG. 7). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, one or more weights are determined based on the domain code. The one or more weights are for weighting one or more parameters of a machine learning based encoder (utilized at step 108 of FIG. 1). The one or more weights may be represented as vectors (or in any other suitable form). In one embodiment, the one or more weights comprise a weight vector dW and a bias vector dB for updating the weight parameter and the bias parameter of the machine learning based encoder.

The one or more weights may be determined using any suitable approach. In one embodiment, the one or more weights are determined using a learnable linear projector. For example, as shown in workflow 200 of FIG. 2, weights 210 are determined by linear projector 208 based on domain code 206. A linear projector is a linear transformation learned during the training process. The linear projector projects or maps the domain code to the one or more weights through a set of parameters optimized during the training process.

At step 106 of FIG. 1, one or more parameters of a machine learning based encoder are updated based on the one or more weights. In one embodiment, the machine learning based encoder is a convolutional layer of a neural network, such as, e.g., a DFN. However, the machine learning based encoder may be any other suitable encoder.

In one embodiment, the one or more parameters of the machine learning based encoder comprise a weight parameter W and a bias parameter B. For example, as shown in workflow 200 of FIG. 2, weight parameter W and bias parameter B of encoder 212 are updated based on weights 210. In one embodiment the one or more parameters are updated by calculating the dot product. For example, the weight parameter may be updated by calculating the dot product of the weight parameter W and the weight vector dW (i.e., W=W·dW) and the bias parameter may be updated by calculating the dot product of the bias parameter B and the bias vector dB (i.e., B=B·dB). Thus, the domain code controls the behavior of the machine learning based encoder. The one or more parameters may be updated according to any other suitable approach.

At step 108 of FIG. 1, features are extracted from the one or more medical images using the machine learning based encoder with the one or more updated parameters. The one or more medical images are concatenated along the channel dimension (together with the all-zero tensors representing medical images of absent domains). The machine learning based encoder (with the one or more updated parameters) receives as input the concatenation and generates as output the features. The features are a lower-dimensional, compressed representation of the one or more medical images represented as a feature vector. In one example, as shown in workflow 200 of FIG. 2, encoder 212 extracts feature map 214 from a concatenation of medical images 202. Consequently, each domain combination of the one or more medical images dynamically updates the machine learning based encoder based on the specific missing domain(s) as defined by the domain code, thereby enabling the extraction of relevant features.

At step 110 of FIG. 1, a medical imaging analysis task is performed based on the extracted features. The medical imaging analysis task may be performed using a machine learning based task network, such as, e.g., a decoder network. The machine learning based task network receives as input the extracted features and generates as output results of the medical imaging analysis task. In one embodiment, the medical imaging analysis task is image synthesis for generating a synthetic image from the one or more medical images. The synthetic image may be in a domain of the set of predefined domains that is absent from the different domains (of the one or more medical images). The medical imaging analysis task may additionally or alternatively comprise any other suitable task, such as, e.g., detection, segmentation, classification, quantification, etc.

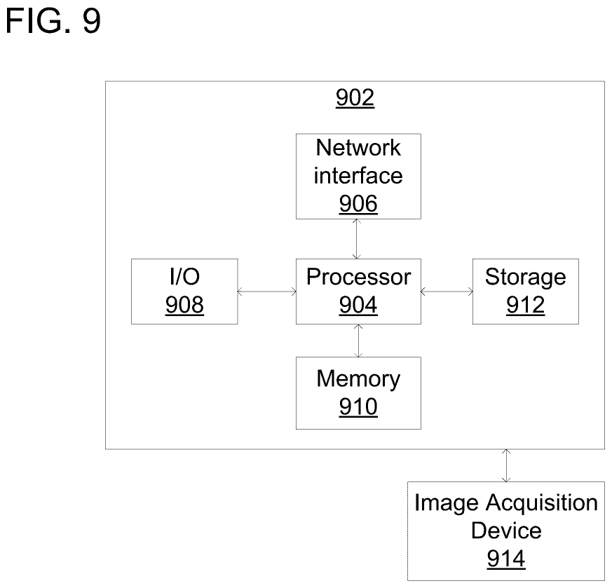

At step 112 of FIG. 1, results of the medical imaging analysis task are output. For example, the results of the medical imaging analysis task can be output by displaying the results on a display device of a computer system (e.g., I/O 908 of computer 902 of FIG. 9), storing the results on a memory or storage of a computer system (e.g., memory 910 or storage 912 of computer 902 of FIG. 9), or by transmitting the results to a remote computer system (e.g., computer 902 of FIG. 9).

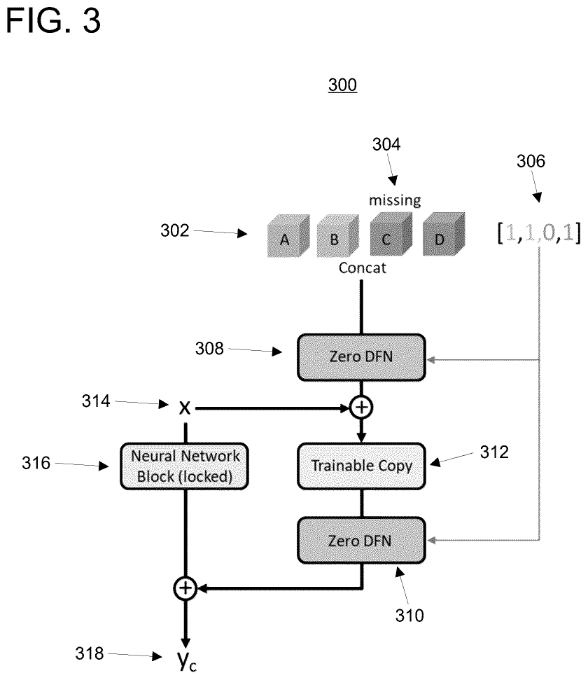

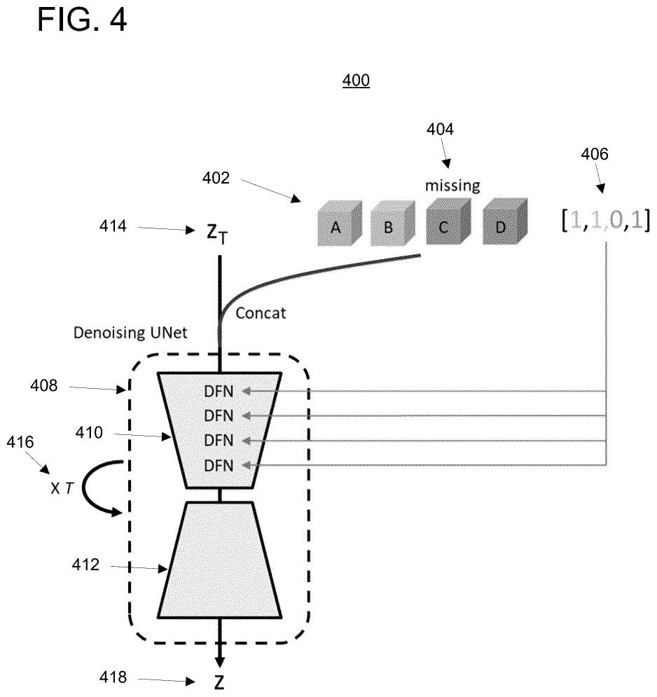

In some embodiments, the machine learning based encoder of FIGS. 1 and 2 may be implemented in a diffusion model for performing a medical imaging analysis task conditioned on multi-domain medical images with missing domains, as shown in FIGS. 3 and 4.

FIG. 3 shows a network architecture 300 of a DFN ControlNet conditioned on multi-domain medical images with missing domains, in accordance with one or more embodiments. Medical images 302 in domains A, B, and D are received and concatenated together with an all-zero tensor 304 for absent or missing domain C. Domain code 306 defines the presence of the one or more medical images 302 in domains A, B, and C and the absence of a medical image in domain C. One or more weights are determined from domain code 306 using a linear projector (not shown in FIG. 3) for updating parameters of zero DFN layers 308 and 310. Zero DFN layers 308 and 310 may be implemented as the machine learning based encoder of step 108 of FIG. 1 or encoder 212 of FIG. 2. Zero DFN layer 308 receives as input the concatenated medical images and generates as output a first set of features. The first set of features are combined with noise X 314 and fed into trainable copy 312, which is a trainable copy of neural network block 316. Neural network block 316 may be, e.g., a resnet block, conv-bn-relu block, multi-head attention block, transformer block, etc. The output of trainable copy 312 is fed into zero DFN layer 310, which generates as output a second set of features. Neural network block 316 receives as input noise X 314 and the output is combined with the second set of features to generate results Y_{C} 318. Advantageously, network architecture 300 enables conditioning on multi-domain medical images and dynamically adjusting its behavior to accommodate different missing domain scenarios.

FIG. 4 shows a network architecture 400 of a DFN DDPM/LDM (denoising diffusion probabilistic model/latent diffusion model) conditioned on multi-domain medical images with missing domains, in accordance with one or more embodiments. Medical images 402 in domains A, B, and D are received and concatenated together with an all-zero tensor 404 for absent or missing domain C. Domain code 406 defines the presence of the one or more medical images 402 in domains A, B, and C and the absence of a medical image in domain C. Network architecture 400 comprises a denoising UNet 408 comprising an encoder 410 and a decoder 412. Encoder 410 comprises a plurality of DFN layers. Each DFN layer may be implemented as the machine learning based encoder of step 108 of FIG. 1 or encoder 212 of FIG. 2. One or more weights are determined from domain code 406 using a linear projector (not shown in FIG. 4) for updating parameters of the DFN layers of encoder 410. The concatenated medical images are combined with noise Z_{T} 414 and fed into encoder 410. The first DFN layer of encoder 410 receives as input a combination of the concatenated medical images and noise Z_{T} 414 and generates as output features. Each subsequent DFN layer receives as input the features output of the prior DFN layer and generates as output features. Decoder 412 receives as input the features output by the last DFN layer of encoder 410 and generates as output results Z 418. In one embodiment, for example where network architecture 400 is of a DFN LDM, medical images 402 are first encoded by encoder 410 to extract features and the features are concatenated along the channel dimension before being fed into the first DFN layer. Advantageously, network architecture 400 enables conditioning on multi-domain medical images.

In one embodiment, for the medical imaging analysis task of synthesizing medical images with a pathology, in addition to the multi-domain medical images, one or more masks of the pathology/lesion to be synthesized and/or of the surrounding tissue or organs may also be received (at step 102). The one or more masks are concatenated along the channel dimension along with the one or more medical images, and features are extracted from the concatenation (at step 108). Method 100 thus continues for performing a medical imaging analysis task based on the extracted features. If not all masks will always be present, the domain code may have corresponding positions defining the presence or absence of the masks. In this way, a multi-domain dataset with pathology can be built with the image synthesis framework in accordance with embodiments described herein.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the linear projector utilized at step 104 or the machine learning based encoder utilized at step 108 of FIG. 1, linear projector 208 or encoder 212 of FIG. 2, zero DFN layers 308 and 310, neural network block 316, or trainable copy 312 of FIG. 3, and/or denoising UNet 408 of FIG. 4, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

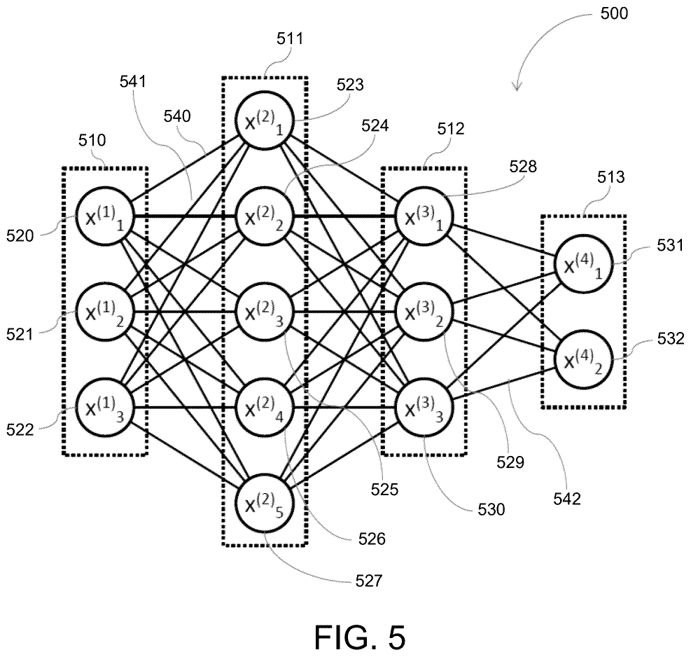

FIG. 5 shows an embodiment of an artificial neural network 500 that may be used to implement one or more machine learning models described herein. Alternative terms for “artificial neural network” are “neural network”, “artificial neural net” or “neural net”.

The artificial neural network 500 comprises nodes 520, . . . , 532 and edges 540, . . . , 542, wherein each edge 540, . . . , 542 is a directed connection from a first node 520, . . . , 532 to a second node 520, . . . , 532. In general, the first node 520, . . . , 532 and the second node 520, . . . 532 are different nodes 520, . . . , 532, it is also possible that the first node 520, . . . , 532 and the second node 520, . . . , 532 are identical. For example, in FIG. 5 the edge 540 is a directed connection from the node 520 to the node 523, and the edge 542 is a directed connection from the node 530 to the node 532. An edge 540, . . . , 542 from a first node 520, . . . , 532 to a second node 520, . . . , 532 is also denoted as “ingoing edge” for the second node 520, . . . , 532 and as “outgoing edge” for the first node 520, . . . , 532.

In this embodiment, the nodes 520, . . . , 532 of the artificial neural network 500 can be arranged in layers 510, . . . , 513, wherein the layers can comprise an intrinsic order introduced by the edges 540, . . . , 542 between the nodes 520, . . . , 532. In particular, edges 540, . . . , 542 can exist only between neighboring layers of nodes. In the displayed embodiment, there is an input layer 510 comprising only nodes 520, . . . , 522 without an incoming edge, an output layer 513 comprising only nodes 531, 532 without outgoing edges, and hidden layers 511, 512 in-between the input layer 510 and the output layer 513. In general, the number of hidden layers 511, 512 can be chosen arbitrarily. The number of nodes 520, . . . , 522 within the input layer 510 usually relates to the number of input values of the neural network, and the number of nodes 531, 532 within the output layer 513 usually relates to the number of output values of the neural network.

In particular, a (real) number can be assigned as a value to every node 520, . . . , 532 of the neural network 500. Here, x⁽ⁿ⁾ᵢ denotes the value of the i-th node 520, . . . , 532 of the n-th layer 510, . . . , 513. The values of the nodes 520, . . . , 522 of the input layer 510 are equivalent to the input values of the neural network 500, the values of the nodes 531, 532 of the output layer 513 are equivalent to the output value of the neural network 500. Furthermore, each edge 540, . . . , 542 can comprise a weight being a real number, in particular, the weight is a real number within the interval [−1, 1] or within the interval [0, 1]. Here, w^{(m,n)}_{i,j} denotes the weight of the edge between the i-th node 520, . . . , 532 of the m-th layer 510, . . . , 513 and the j-th node 520, . . . , 532 of the n-th layer 510, . . . , 513. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 520, . . . , 532 of the (n+1)-th layer 510, . . . , 513 can be calculated based on the values of the nodes 520, . . . , 532 of the n-th layer 510, . . . , 513 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 510 are given by the input of the neural network 500, wherein values of the first hid-den layer 511 can be calculated based on the values of the input layer 510 of the neural network, wherein values of the second hidden layer 512 can be calculated based in the values of the first hidden layer 511, etc.

In order to set the values w^{(m,n)}_{i,j} for the edges, the neural network 500 has to be trained using training data. In particular, training data comprises training input data and training output data (denoted as tᵢ). For a training step, the neural network 500 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 513, wherein f′ is the first derivative of the activation function, and t (n+1); is the comparison training value for the j-th node of the output layer 513.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

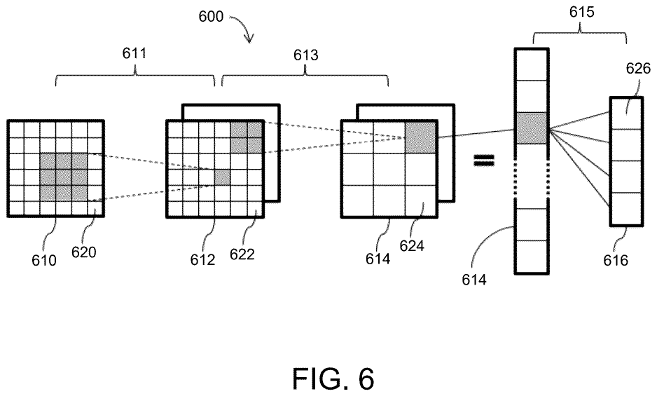

FIG. 6 shows an embodiment of a convolutional neural network 600 that may be used to implement one or more machine learning models described herein. In the displayed embodiment, the convolutional neural network 600 comprises an input node layer 610, a convolutional layer 611, a pooling layer 613, a fully connected layer 614 and an output node layer 616, as well as hidden node layers 612, 614. Alternatively, the convolutional neural network 600 can comprise several convolutional layers 611, several pooling layers 613 and several fully connected layers 615, as well as other types of layers. The order of the layers can be chosen arbitrarily, usually fully connected layers 615 are used as the last layers before the output layer 616.

In particular, within a convolutional neural network 600 nodes 620, 622, 624 of a node layer 610, 612, 614 can be considered to be arranged as a d-dimensional matrix or as a d-dimensional image. In particular, in the two-dimensional case the value of the node 620, 622, 624 indexed with i and j in the n-th node layer 610, 612, 614 can be denoted as x(n)[i, j]. However, the arrangement of the nodes 620, 622, 624 of one node layer 610, 612, 614 does not have an effect on the calculations executed within the convolutional neural network 600 as such, since these are given solely by the structure and the weights of the edges.

A convolutional layer 611 is a connection layer between an anterior node layer 610 (with node values x(n−1)) and a posterior node layer 612 (with node values x(n)). In particular, a convolutional layer 611 is characterized by the structure and the weights of the incoming edges forming a convolution operation based on a certain number of kernels. In particular, the structure and the weights of the edges of the convolutional layer 611 are chosen such that the values x(n) of the nodes 622 of the posterior node layer 612 are calculated as a convolution x(n)=K*x(n−1) based on the values x(n−1) of the nodes 620 anterior node layer 610, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 620, 622 (e.g., a 3×3 matrix, or a 5×5 matrix). In particular, this implies that the weights of the edges in the convolution layer 611 are not independent, but chosen such that they produce said convolution equation. In particular, for a kernel being a 3×3 matrix, there are only 9 independent weights (each entry of the kernel matrix corresponding to one independent weight), irrespectively of the number of nodes 620, 622 in the anterior node layer 610 and the posterior node layer 612.

In general, convolutional neural networks 600 use node layers 610, 612, 614 with a plurality of channels, in particular, due to the use of a plurality of kernels in convolutional layers 611. In those cases, the node layers can be considered as (d+1)-dimensional matrices (the first dimension indexing the channels). The action of a convolutional layer 611 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ₐ corresponds to the a-th channel of the anterior node layer 610, x⁽ⁿ⁾_{b} corresponds to the b-th channel of the posterior node layer 612 and K_{a,b} corresponds to one of the kernels. If a convolutional layer 611 acts on an anterior node layer 610 with A channels and outputs a posterior node layer 612 with B channels, there are A·B independent d-dimensional kernels K_{a,b}.

In general, in convolutional neural networks 600 activation functions are used. In this embodiment ReLU (acronym for “Rectified Linear Units”) is used, with R(z)=max(0, z), so that the action of the convolutional layer 611 in the two-dimensional example is

$${x}^{{( n )}_{b}} [ i , j ] = ( {\sum }_{a} {K}_{a , b} * {x}^{{( n - 1 )}_{a}} [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 610 comprises 36 nodes 620, arranged as a two-dimensional 6×6 matrix. The first hidden node layer 612 comprises 72 nodes 622, arranged as two two-dimensional 6×6 matrices, each of the two matrices being the result of a convolution of the values of the input layer with a 3×3 kernel within the convolutional layer 611. Equivalently, the nodes 622 of the first hidden node layer 612 can be interpreted as arranged as a three-dimensional 2×6×6 matrix, wherein the first dimension correspond to the channel dimension.

The advantage of using convolutional layers 611 is that spatially local correlation of the input data can exploited by enforcing a local connectivity pattern between nodes of adjacent layers, in particular by each node being connected to only a small region of the nodes of the preceding layer.

A pooling layer 613 is a connection layer between an anterior node layer 612 (with node values x(n−1)) and a posterior node layer 614 (with node values x(n)). In particular, a pooling layer 613 can be characterized by the structure and the weights of the edges and the activation function forming a pooling operation based on a non-linear pooling function f. For example, in the two-dimensional case the values x(n) of the nodes 624 of the posterior node layer 614 can be calculated based on the values x(n−1) of the nodes 622 of the anterior node layer 612 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 613 the number of nodes 622, 624 can be reduced, by re-placing a number d1·d2 of neighboring nodes 622 in the anterior node layer 612 with a single node 622 in the posterior node layer 614 being calculated as a function of the values of said number of neighboring nodes. In particular, the pooling function f can be the max-function, the average or the L2-Norm. In particular, for a pooling layer 613 the weights of the incoming edges are fixed and are not modified by training.

The advantage of using a pooling layer 613 is that the number of nodes 622, 624 and the number of parameters is reduced. This leads to the amount of computation in the network being reduced and to a control of overfitting.

In the displayed embodiment, the pooling layer 613 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 600 are fully connected layers 615. A fully connected layer 615 is a connection layer between an anterior node layer 614 and a posterior node layer 616. A fully connected layer 613 can be characterized by the fact that a majority, in particular, all edges between nodes 614 of the anterior node layer 614 and the nodes 616 of the posterior node layer are present, and wherein the weight of each of these edges can be adjusted individually.

In this embodiment, the nodes 624 of the anterior node layer 614 of the fully connected layer 615 are displayed both as two-dimensional matrices, and additionally as non-related nodes (indicated as a line of nodes, wherein the number of nodes was reduced for a better presentability). This operation is also denoted as “flattening”. In this embodiment, the number of nodes 626 in the posterior node layer 616 of the fully connected layer 615 smaller than the number of nodes 624 in the anterior node layer 614. Alternatively, the number of nodes 626 can be equal or larger.

Furthermore, in this embodiment the Softmax activation function is used within the fully connected layer 615. By applying the Softmax function, the sum the values of all nodes 626 of the output layer 616 is 1, and all values of all nodes 626 of the output layer 616 are real numbers between 0 and 1. In particular, if using the convolutional neural network 600 for categorizing input data, the values of the output layer 616 can be interpreted as the probability of the input data falling into one of the different categories.

In particular, convolutional neural networks 600 can be trained based on the backpropagation algorithm. For preventing overfitting, methods of regularization can be used, e.g., dropout of nodes 620, . . . , 624, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

FIG. 7 shows a data flow diagram according to an embodiment for using a generative adversarial network for creating synthetic output data G(x) 708 based on input data x 702 that is indistinguishable from real output data y 704, in accordance with one or more embodiments. The synthetic output data G(x) 708 has the same structure as the real output data y 704, but its content is not derived from real world data.

The generative adversarial network comprises a generator function G 706 and a classifier function C 710 which are trained jointly. The task of the generator function G 706 is to provide realistic synthetic output data G(x) 708 based on input data x 702, and the task of the classifier function C 710 is to distinguish between real output data y 704 and synthetic output data G(x) 708. In particular, the output of the classifier function C 710 is a real number between 0 and 1 corresponding to the probability of the input value being real data, so that an ideal classifier function would calculate an output value of C(y) 714≈1 for real data y 704 and C(G(x)) 712≈0 for synthetic data G(x) 708.

Within the training process, parameters of the generator function G 706 are adapted so that the synthetic output data G(x) 708 has the same characteristics as real output data y 704, so that the classifier function C 710 cannot distinguish between real and synthetic data anymore. At the same time, parameters of the classifier function C 710 are adapted so that it distinguishes between real and synthetic data in the best possible way. Here, the training relies on pairs comprising input data x 702 and the corresponding real output data y 704. Within a single training step, the generator function G 706 is applied to the input data x 702 for generating synthetic output data G(x) 708. Furthermore, the classifier function C 710 is applied to the real output data y 704 for generating a first classification result C(y) 714. Additionally, the classifier function C 710 is applied to the synthetic output data G(x) 708 for generating a second classification result C (G(x)) 712.

Adapting the parameters of the generative function G 706 and the classifier function C 710 is based on minimizing a cost function by using the backpropagation algorithm, respectively. In this embodiment, the cost function K_{C} for the classifier function C 710 is K_{C}∝−BCE(C(y), 1)−BCE(C(G(x), 0), wherein BCE denotes the binary cross entropy defined as BCE(z, z′)=z′·log(z)+(1−z′)·log(1−z). By using this cost function, both wrongly classifying real output data as synthetic (indicated by C(y)=0) and wrongly classifying synthetic output data as real (indicated as C(G(x)) 712≈1) increases the cost function K_{C} to be minimized. Furthermore, the cost function K_{G} for the generator function G 706 is K_{G}∝−BCE(C(G(x), 1)=−log(C(G(x). By using this cost function, correctly classified synthetic output data (indicated as C(G(x)) 712≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

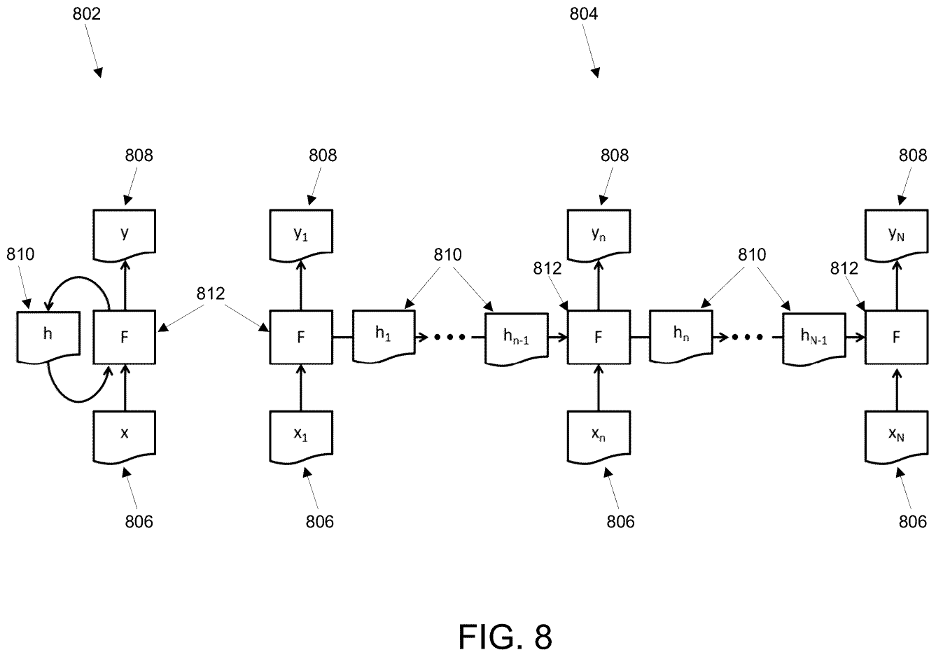

FIG. 8 shows the schematic structure of a recurrent machine learning model F, both in a recurrent representation 802 and in an unfolded representation 804, that may be used to implement one or more machine learning models described herein. The recurrent machine learning model takes as input several input datasets x, x₁, . . . , x_{N} 806 and creates a corresponding set of output datasets y, y₁, . . . , y_{N} 808. Furthermore, the output depends on a so-called hidden vector h, h₁, . . . , h_{N} 810, which implicitly comprises information about input datasets previously used as input for the recurrent machine learning model F 812. By using these hidden vectors h, h₁, . . . , h_{N} 810, a sequentiality of the input datasets can be leveraged.

In a single step of the processing, the recurrent machine learning model F 812 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(Xₙ, hₙ₋₁), or by splitting the recurrent machine learning model F 812 into a part F(y) calculating the output data and F (h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 812 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F^{(h)}(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 902 that may be used to implement systems, apparatuses, and methods described herein is depicted in FIG. 9. Computer 902 includes a processor 904 operatively coupled to a data storage device 912 and a memory 910. Processor 904 controls the overall operation of computer 902 by executing computer program instructions that define such operations. The computer program instructions may be stored in data storage device 912, or other computer readable medium, and loaded into memory 910 when execution of the computer program instructions is desired. Thus, the method and workflow steps or functions of FIGS. 1-3 can be defined by the computer program instructions stored in memory 910 and/or data storage device 912 and controlled by processor 904 executing the computer program instructions. For example, the computer program instructions can be implemented as computer executable code programmed by one skilled in the art to perform the method and workflow steps or functions of FIGS. 1-3. Accordingly, by executing the computer program instructions, the processor 904 executes the method and workflow steps or functions of FIGS. 1-3. Computer 902 may also include one or more network interfaces 906 for communicating with other devices via a network. Computer 902 may also include one or more input/output devices 908 that enable user interaction with computer 902 (e.g., display, keyboard, mouse, speakers, buttons, etc.).

Processor 904 may include both general and special purpose microprocessors, and may be the sole processor or one of multiple processors of computer 902. Processor 904 may include one or more central processing units (CPUs), for example. Processor 904, data storage device 912, and/or memory 910 may include, be supplemented by, or incorporated in, one or more application-specific integrated circuits (ASICs) and/or one or more field programmable gate arrays (FPGAs).

Data storage device 912 and memory 910 each include a tangible non-transitory computer readable storage medium. Data storage device 912, and memory 910, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 908 may include peripherals, such as a printer, scanner, display screen, etc. For example, input/output devices 908 may include a display device such as a cathode ray tube (CRT) or liquid crystal display (LCD) monitor for displaying information to the user, a keyboard, and a pointing device such as a mouse or a trackball by which the user can provide input to computer 902.

An image acquisition device 914 can be connected to the computer 902 to input image data (e.g., medical images) to the computer 902. It is possible to implement the image acquisition device 914 and the computer 902 as one device. It is also possible that the image acquisition device 914 and the computer 902 communicate wirelessly through a network. In a possible embodiment, the computer 902 can be located remotely with respect to the image acquisition device 914.

Any or all of the systems, apparatuses, and methods discussed herein may be implemented using one or more computers such as computer 902.

One skilled in the art will recognize that an implementation of an actual computer or computer system may have other structures and may contain other components as well, and that FIG. 9 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) one or more medical images each in a different domain and 2) a domain code defining a presence of the different domains in a set of predefined domains; determining one or more weights based on the domain code; updating one or more parameters of a machine learning based encoder based on the one or more weights; extracting features from the one or more medical images using the machine learning based encoder with the one or more updated parameters; performing a medical imaging analysis task based on the extracted features; and outputting results of the medical imaging analysis task.

Illustrative embodiment 2. The computer-implemented method of illustrative embodiment 1, wherein the domain code further defines an absence of one or more domains of the set of predefined domains from the different domains.

Illustrative embodiment 3. The computer-implemented method of any one of illustrative embodiments 1-2, wherein each position of the domain code is associated with a respective one of the set of predefined domains.

Illustrative embodiment 4. The computer-implemented method of any one of illustrative embodiments 1-3, wherein determining one or more weights based on the domain code comprises: projecting the domain code to the one or more weights using a linear projector.

Illustrative embodiment 5. The computer-implemented method of any one of illustrative embodiments 1-4, wherein updating one or more parameters of a machine learning based encoder based on the one or more weights comprises: updating a weight parameter and a bias parameter of the machine learning based encoder.

Illustrative embodiment 6. The computer-implemented method of any one of illustrative embodiments 1-5, wherein updating one or more parameters of a machine learning based encoder based on the one or more weights comprises: determining a dot product of the one or more parameters of the machine learning based encoder and a respective one of the one or more weights.

Illustrative embodiment 7. The computer-implemented method of any one of illustrative embodiments 1-6, wherein: receiving 1) one or more medical images each in a different domain and 2) a domain code defining a presence of the different domains in a set of predefined domains comprises: receiving one or more all-zero tensors for one or more domains of the set of predefined domains absent from the different domains; and extracting features from the one or more medical images using the machine learning based encoder with the one or more updated parameters comprises: concatenating the one or more medical images with the one or more all-zero tensors, and extracting features from the concatenation.

Illustrative embodiment 8. The computer-implemented method of any one of illustrative embodiments 1-7, wherein: receiving 1) one or more medical images each in a different domain and 2) a domain code defining a presence of the different domains in a set of predefined domains comprises: receiving one or more masks of at least one of a pathology or an organ; and extracting features from the one or more medical images using the machine learning based encoder with the one or more updated parameters comprises: concatenating the one or more medical images with the one or more masks, and extracting features from the concatenation.

Illustrative embodiment 9. The computer-implemented method of any one of illustrative embodiments 1-8, wherein the medical imaging analysis task comprises medical image synthesis.

Illustrative embodiment 10. An apparatus comprising: means for receiving 1) one or more medical images each in a different domain and 2) a domain code defining a presence of the different domains in a set of predefined domains; means for determining one or more weights based on the domain code; means for updating one or more parameters of a machine learning based encoder based on the one or more weights; means for extracting features from the one or more medical images using the machine learning based encoder with the one or more updated parameters; means for performing a medical imaging analysis task based on the extracted features; and means for outputting results of the medical imaging analysis task.

Illustrative embodiment 11. The apparatus of illustrative embodiment 10, wherein the domain code further defines an absence of one or more domains of the set of predefined domains from the different domains.

Illustrative embodiment 12. The apparatus of any one of illustrative embodiments 10-11, wherein each position of the domain code is associated with a respective one of the set of predefined domains.

Illustrative embodiment 13. The apparatus of any one of illustrative embodiments 10-12, wherein the means for determining one or more weights based on the domain code comprises: means for projecting the domain code to the one or more weights using a linear projector.

Illustrative embodiment 14. The apparatus of any one of illustrative embodiments 10-13, wherein the means for updating one or more parameters of a machine learning based encoder based on the one or more weights comprises: means for updating a weight parameter and a bias parameter of the machine learning based encoder.

Illustrative embodiment 15. A non-transitory computer-readable storage medium comprising instructions which, when executed by a computer, cause the computer to carry out operations comprising: receiving 1) one or more medical images each in a different domain and 2) a domain code defining a presence of the different domains in a set of predefined domains; determining one or more weights based on the domain code; updating one or more parameters of a machine learning based encoder based on the one or more weights; extracting features from the one or more medical images using the machine learning based encoder with the one or more updated parameters; performing a medical imaging analysis task based on the extracted features; and outputting results of the medical imaging analysis task.

Illustrative embodiment 16. The non-transitory computer-readable storage medium of illustrative embodiment 15, wherein the domain code further defines an absence of one or more domains of the set of predefined domains from the different domains.

Illustrative embodiment 17. The non-transitory computer-readable storage medium of any one of illustrative embodiments 15-16, wherein updating one or more parameters of a machine learning based encoder based on the one or more weights comprises: determining a dot product of the one or more parameters of the machine learning based encoder and a respective one of the one or more weights.

Illustrative embodiment 18. The non-transitory computer-readable storage medium of any one of illustrative embodiments 15-17, wherein: receiving 1) one or more medical images each in a different domain and 2) a domain code defining a presence of the different domains in a set of predefined domains comprises: receiving one or more all-zero tensors for one or more domains of the set of predefined domains absent from the different domains; and extracting features from the one or more medical images using the machine learning based encoder with the one or more updated parameters comprises: concatenating the one or more medical images with the one or more all-zero tensors, and extracting features from the concatenation.

Illustrative embodiment 19. The non-transitory computer-readable storage medium of any one of illustrative embodiments 15-18, wherein: receiving 1) one or more medical images each in a different domain and 2) a domain code defining a presence of the different domains in a set of predefined domains comprises: receiving one or more masks of at least one of a pathology or an organ; and extracting features from the one or more medical images using the machine learning based encoder with the one or more updated parameters comprises: concatenating the one or more medical images with the one or more masks, and extracting features from the concatenation.

Illustrative embodiment 20. The non-transitory computer-readable storage medium of any one of illustrative embodiments 15-19, wherein the medical imaging analysis task comprises medical image synthesis.

1. A computer-implemented method comprising:
receiving 1) one or more medical images each in a different domain and 2) a domain code defining a presence of the different domains in a set of predefined domains;
determining one or more weights based on the domain code;
updating one or more parameters of a machine learning based encoder based on the one or more weights;
extracting features from the one or more medical images using the machine learning based encoder with the one or more updated parameters;
performing a medical imaging analysis task based on the extracted features; and
outputting results of the medical imaging analysis task.

2. The computer-implemented method of claim 1, wherein the domain code further defines an absence of one or more domains of the set of predefined domains from the different domains.

3. The computer-implemented method of claim 1, wherein each position of the domain code is associated with a respective one of the set of predefined domains.

4. The computer-implemented method of claim 1, wherein determining one or more weights based on the domain code comprises:
projecting the domain code to the one or more weights using a linear projector.

5. The computer-implemented method of claim 1, wherein updating one or more parameters of a machine learning based encoder based on the one or more weights comprises:
updating a weight parameter and a bias parameter of the machine learning based encoder.

6. The computer-implemented method of claim 1, wherein updating one or more parameters of a machine learning based encoder based on the one or more weights comprises:
determining a dot product of the one or more parameters of the machine learning based encoder and a respective one of the one or more weights.

7. The computer-implemented method of claim 1, wherein:
receiving 1) one or more medical images each in a different domain and 2) a domain code defining a presence of the different domains in a set of predefined domains comprises:
receiving one or more all-zero tensors for one or more domains of the set of predefined domains absent from the different domains; and
extracting features from the one or more medical images using the machine learning based encoder with the one or more updated parameters comprises:
concatenating the one or more medical images with the one or more all-zero tensors, and
extracting features from the concatenation.

8. The computer-implemented method of claim 1, wherein:
receiving 1) one or more medical images each in a different domain and 2) a domain code defining a presence of the different domains in a set of predefined domains comprises:
receiving one or more masks of at least one of a pathology or an organ; and
extracting features from the one or more medical images using the machine learning based encoder with the one or more updated parameters comprises:
concatenating the one or more medical images with the one or more masks, and
extracting features from the concatenation.

9. The computer-implemented method of claim 1, wherein the medical imaging analysis task comprises medical image synthesis.

10. An apparatus comprising:
means for receiving 1) one or more medical images each in a different domain and 2) a domain code defining a presence of the different domains in a set of predefined domains;
means for determining one or more weights based on the domain code;
means for updating one or more parameters of a machine learning based encoder based on the one or more weights;
means for extracting features from the one or more medical images using the machine learning based encoder with the one or more updated parameters;
means for performing a medical imaging analysis task based on the extracted features; and
means for outputting results of the medical imaging analysis task.

11. The apparatus of claim 10, wherein the domain code further defines an absence of one or more domains of the set of predefined domains from the different domains.

12. The apparatus of claim 10, wherein each position of the domain code is associated with a respective one of the set of predefined domains.

13. The apparatus of claim 10, wherein the means for determining one or more weights based on the domain code comprises:
means for projecting the domain code to the one or more weights using a linear projector.

14. The apparatus of claim 10, wherein the means for updating one or more parameters of a machine learning based encoder based on the one or more weights comprises:
means for updating a weight parameter and a bias parameter of the machine learning based encoder.

15. A non-transitory computer-readable storage medium comprising instructions which, when executed by a computer, cause the computer to carry out operations comprising:
receiving 1) one or more medical images each in a different domain and 2) a domain code defining a presence of the different domains in a set of predefined domains;
determining one or more weights based on the domain code;
updating one or more parameters of a machine learning based encoder based on the one or more weights;
extracting features from the one or more medical images using the machine learning based encoder with the one or more updated parameters;
performing a medical imaging analysis task based on the extracted features; and
outputting results of the medical imaging analysis task.

16. The non-transitory computer-readable storage medium of claim 15, wherein the domain code further defines an absence of one or more domains of the set of predefined domains from the different domains.

17. The non-transitory computer-readable storage medium of claim 15, wherein updating one or more parameters of a machine learning based encoder based on the one or more weights comprises:
determining a dot product of the one or more parameters of the machine learning based encoder and a respective one of the one or more weights.

18. The non-transitory computer-readable storage medium of claim 15, wherein:
receiving 1) one or more medical images each in a different domain and 2) a domain code defining a presence of the different domains in a set of predefined domains comprises:
receiving one or more all-zero tensors for one or more domains of the set of predefined domains absent from the different domains; and
extracting features from the one or more medical images using the machine learning based encoder with the one or more updated parameters comprises:
concatenating the one or more medical images with the one or more all-zero tensors, and
extracting features from the concatenation.

19. The non-transitory computer-readable storage medium of claim 15, wherein:
receiving 1) one or more medical images each in a different domain and 2) a domain code defining a presence of the different domains in a set of predefined domains comprises:
receiving one or more masks of at least one of a pathology or an organ; and
extracting features from the one or more medical images using the machine learning based encoder with the one or more updated parameters comprises:
concatenating the one or more medical images with the one or more masks, and
extracting features from the concatenation.

20. The non-transitory computer-readable storage medium of claim 15, wherein the medical imaging analysis task comprises medical image synthesis.
